(19) United States
(12) Patent Application Publication
(10) Pub. No.: US 2015/0191288 A1
(43) Pub. Date: Jul. 9, 2015

(54) BLISTER PACK

(71) Applicant: Advent Pharmaceuticals Pty Ltd, Notting Hill, Victoria (AU)

(72) Inventor: Nicholas Jolley, Victoria (AU)

(21) Appl. No.: 14/412,926

(22) PCT Filed: Jun. 24, 2013
(86) PCT No.: PCT/AU2013/000675

§ 371 Date: Jan. 5, 2015

(30) Foreign Application Priority Data
Jul. 3, 2012 (AU) 2012902839

(51) Int. Cl. B65D 75/32; A61M 15/00; A61J 1/00

(52) U.S. Cl. CPC B65D 75/327; A61J 1/00; A61M 15/0021; A61M 15/0051; A61M 15/0058; A61J 1/035

(57) ABSTRACT

A blister pack for containing medicament in powder form for use within an inhalation device, the blister pack comprising an elongate strip of base material containing equally spaced recesses along the length of the strip, each recess having walls defining a cavity, and a lid hermetically sealed onto the base material to close the cavities and define pockets of medicament powder, the blister strip being adapted to have each recess filled with a metered dose of medicament powder, each recess being elongate having its longitudinal axis extending across the strip and having longer side walls joined by a base, whereby the volume of each recess is adapted to be greater than the volume of the metered dose and each pocket of the blister strip is adapted to be opened by either pulling back the lid or puncturing the lid or the walls of the recess.

### INTRODUCTION

This invention relates to blister packs which contain medicament in powder form for use within inhalation devices.

### BACKGROUND OF THE INVENTION

Inhalation devices are known for use with blister packs in which the medicament is held in powder form. (e.g. U.S. Pat. No. 5,873,360). Blister packs can comprise an elongate strip formed from a base material having a plurality of recesses spaced along its length and a lid material hermetically sealed thereto to define a plurality of pocket cavities, each pocket cavity having therein inhalable medicament in powder form. The lid material can form a peelable or non-peelable seal to the base material.

Inhalation devices include means for opening the pocket to allow inhalation of the contents. The opening means could peel the material of the blister pocket apart (e.g. U.S. Pat. No. 5,873,360), or consist of a puncturing member to puncture the lid of each pocket, or a cutting means to open the pocket through the lid material or the base material.

U.S. Pat. No. 7,389,775 describes a blister pack which consists of:
The spacing between the pockets can be equi-spaced or spacing increases along the length of the strip
Pockets have a lengthways dimension and a sideways dimension and wherein the lengthways dimension is smaller than the sideways dimension
The lengthways spacing between adjacent pockets is less than the sideways dimension of the pockets
The pockets are elongate in a transverse direction to the strip length.
The blister pack is flexible and can be in roll form.
The pocket has a blind cavity with a closed and open end and a lid over the open end for closure of the blind cavity to retain the medicament powder.

This blister pack interacts with separate indexing and opening mechanisms to peel open the pockets. The blister pocket is designed to maximize the number of doses contained in a blister strip (60-100 doses) without making the inhaler unnecessarily large. While there are other means to contain the size of the inhalation device (e.g. detaching the opened pocket (US 2008/0135441) or crushing the used pocket (US 2009/0007908), limiting the overall length and depth of the blister strip is a simple means.

U.S. Pat. No. 5,187,921 describes a system for filling a blind cavity in a blister pack where the quantity of the medicament powder is controlled by using the dimensions (volume) of the cavity in the base material to be filled as the base material passes through a powder bed. Excess medicament powder on the surface of the base material between the cavities is removed via a series of cleaning stations using doctor blades or rotating teeth synchronized with the movement of the base sheet.

Accurate dosing of the medicament powder into each cavity of the blister strip is an important factor in achieving the required delivered dose performance of an inhalation device. Proposed new United States Pharmacopia criteria for the inhaler delivered dose uniformity testing potentially requires a reduction in delivered dose variability to allow inhalation products to routinely pass the test with a high degree of certainty.

Reducing the variability in the quantity of powder in each of the pockets of the blister strip may be the simplest means to achieve the required reduction in delivered dose variability.

While there are alternate filling systems (eg DE10046127) which can accurately and consistently dose powder into a blister cavity rather than relying of the dimensions of the formed pocket to control the fill weight of the powder, the disadvantage of using these alternate filling systems is that a larger cavity is required to allow for placement of an equivalent weight of dosed powder.

It is these issues that have brought about the present invention.

An object of the present invention is to provide a design of the blister pack which allows a headspace required for accurate dosing of the medicament powder without the blister becoming unacceptably large and impacting on the overall size of the inhalation device.

### SUMMARY OF THE INVENTION

According to one aspect of the present invention there is provided a blister pack for containing medicament in powder form for use within an inhalation device, the blister pack comprising an elongate strip of base material containing spaced recesses along the length of the strip, each recess having walls defining a cavity, and a lid hermetically sealed onto the base material to close the cavities and define pockets for medicament powder, the blister strip being adapted to have each recess filled with a metered dose of medicament powder, each recess being elongate having its longitudinal axis extending across the strip and having longer side walls joined by a base to define a mouth, whereby the volume of each recess is adapted to be greater than the volume of the metered dose and each pocket of the blister strip is adapted to be opened by either pulling back the lid or puncturing the lid or the walls of the recess.

Preferably at least one side includes a laterally extending cavity above the base

In one option, a laterally extending cavity above the base is provided on each side defining a larger mouth and larger cavity without increasing the depth of the cavity.

In another option, the cavity above the base extends around the periphery of the recess.

### DESCRIPTION OF THE DRAWINGS

Embodiments of the present invention will now be described by way of example only with reference to the accompanying drawings in which:

FIG. 1 is a perspective view of a blister pack in accordance with a first embodiment illustrating base material covered by lid material,

FIG. 2 is a plan view of a plurality of cavities in the base material,

FIG. 3 is a cross sectional view taken along the lines D-D of FIG. 2,

FIG. 4 is a cross sectional view taken along the lines C-C of FIG. 2,

FIGS. 5a and 5b are a plan and perspective views of a plurality of cavities in accordance with a second embodiment,

FIG. 6 is a cross sectional view taken along the lines B-B of FIG. 5a,

FIG. 7 is a cross sectional view taken along the lines C-C of FIG. 5a,

FIG. 8 is a cross sectional view taken along the lines D-D of FIG. 5a,

FIG. 9 is a cross sectional view taken along the lines E-E of FIG. 5a,

FIGS. 10a and 10b are plan and perspective views of a plurality of cavities in accordance with a third embodiment,

FIG. 11 is a cross sectional view taken along the lines B-B of FIG. 10a, and

FIG. 12 is a cross sectional view taken along the lines C-C of FIG. 10a.

### DESCRIPTION OF THE PREFERRED EMBODIMENTS

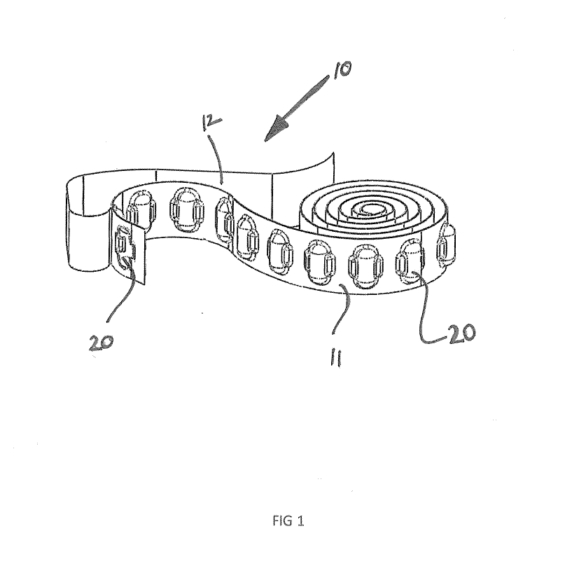

In a first embodiment a blister pack 10 shown in FIGS. 1 to 4 of the accompanying drawings comprising an elongate strip of base material 11 covered by a lid material 12 that is hermetically sealed to the base material. The base material and lid material define an elongate strip which, as shown in FIG. 1, can be wound into a coil. Equally spaced along the length of the base material are a series of recesses 20 that define cavities that are covered by the lid material. The covered recesses 20 define pockets for medicament powder. The blister pack is designed to define between sixty and a hundred sealed cavities each of which is filled with powder.

The base material is preferably a metal foil or polymer laminate material and the recesses 20 are pressed into the base material. The lid 12 is PVC coated with adhesive that secures the lid 12 to the base material 11 to hermetically seal each pocket. The blister pack 10 has been designed so that it can be used in a variety of inhalation devices and as a consequence, it is understood that each pocket can be opened by a variety of means such as peeling the lid off the pocket, as shown in U.S. Pat. No. 5,873,360 or puncturing the lid or the pocket wall to open the pocket. Once the pocket is open the user inhales thereby sucking up an airflow that draws the powder out of the pocket and through the mouthpiece of the inhaler.

Details of each recess are shown in particular detail in FIGS. 2 to 4.

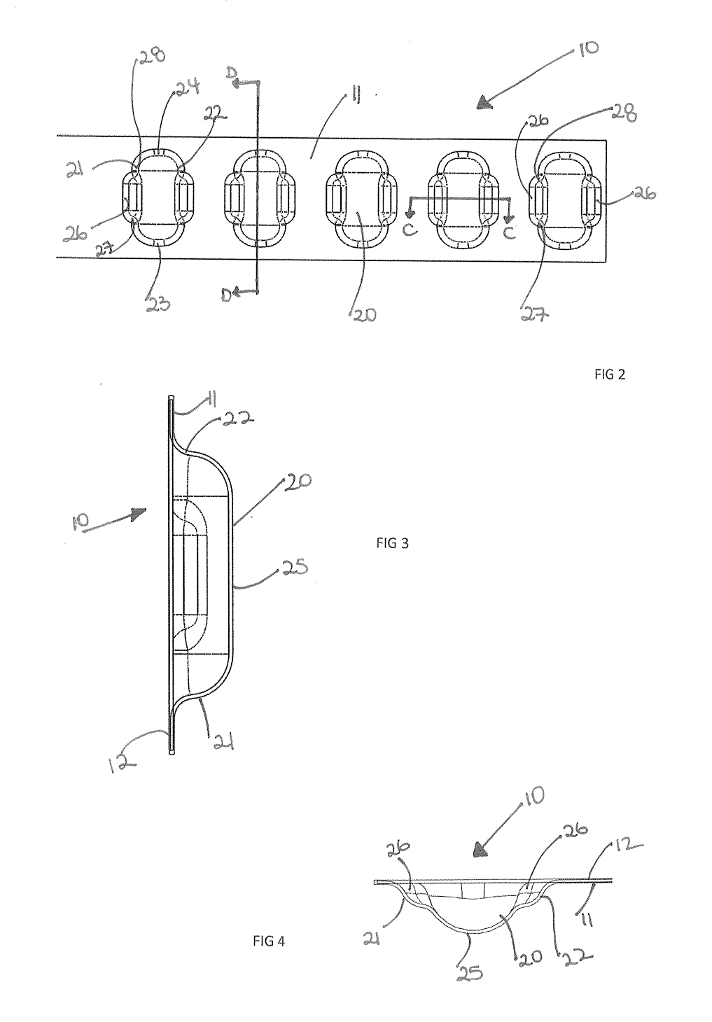

As shown in the plan of FIG. 2, each recess 20 is elongate with its longitudinal axis extending across the strip of base material. The recess has sides 21, 22 joined by slightly curved end walls 23, 24. As shown in FIG. 4 the sides 21, 22 curve downwardly to join a U-shaped base 25. The centre of the recess 20 is the deepest portion of the cavity. Each side wall 21, 22 includes a sheet 26 that defines a supplementary cavity that extends partway along the length of the recess 20. The supplementary cavities 26 increase the width of each recess 20 and define lateral cavities above the base 25 of the recess 20. The lid material 12 as shown in FIG. 3 extends across the main and supplementary cavities recesses to define a closed pocket as shown in FIGS. 3 and 4.

FIGS. 3 and 4 show the curved nature of the side walls 21, 22 through the shelf 26 defining the wing like supplementary cavities on either side of the main recess 20.

The outer angle on the recess walls 21, 22 is designed to match the inhaler gear used to index a blister strip.

Preferably, the width of the blister strip is between 9 and 13 mm. When using an aluminium laminate construction the maximum length of the recess is typically 7 mm, maximum width of the recess is typically 5 mm with at least 2 mm distance between recesses. The maximum depth of the central cavity is 1.5 mm and the depth of the two supplementary cavities is typically 0.5 mm. By creating the two supplementary cavities in the main cavity the volume is increased from 21.45=³ to approximately 26.7=3, that is approximately a 25% increase in volume. The increase volume of the cavity 26.7 mm³ allows a 14.0 mm³ dose of powder to be adequately accommodated within the blister cavity thus minimising splashing of powder onto the surface that is sealed to the lid material.

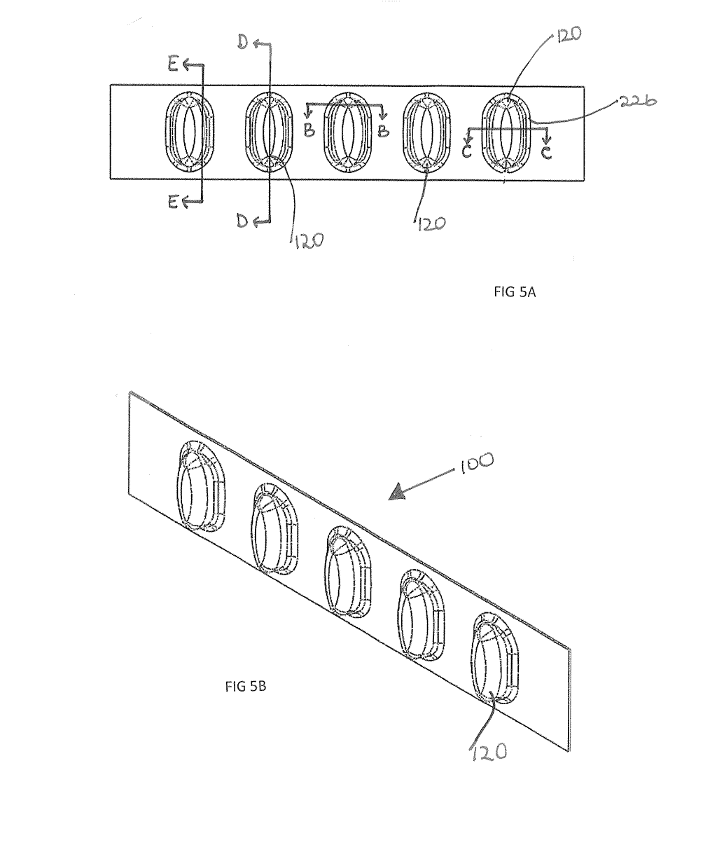

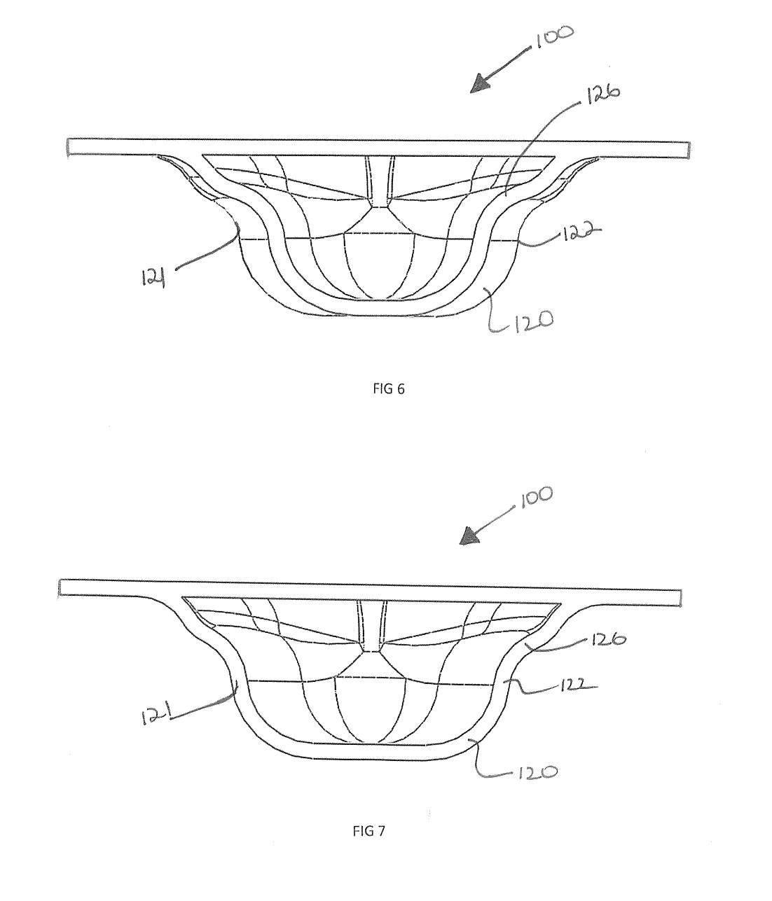

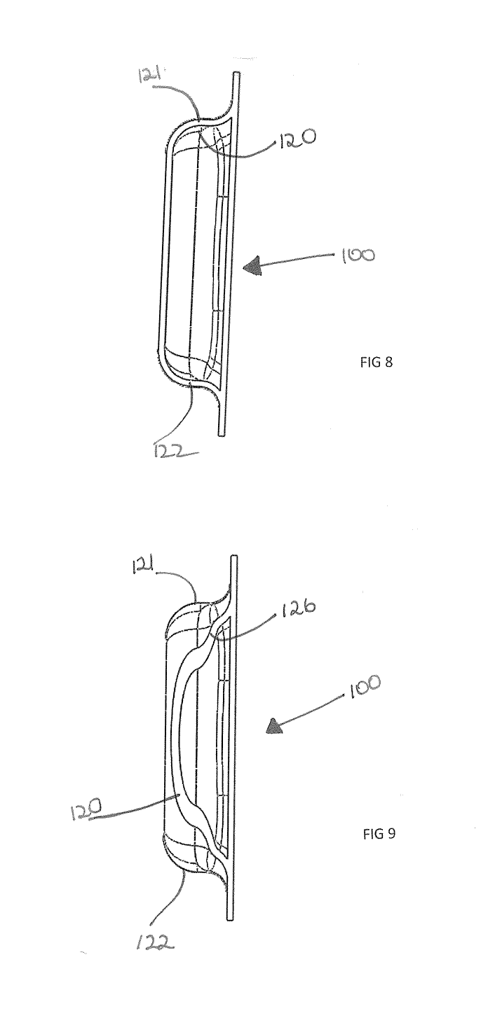

In a second embodiment of the blister strip 100 shown in FIGS. 5 to 9 each recess 120 is elliptically shaped as viewed in plan and has a substantially elliptical shelf 126 approximately three quarters of the way up the side walls 121, 122 of the recess see FIGS. 6 and 7. Unlike the shelf 26 that extends like wings in the first embodiment the shelf 126 in this embodiment extends substantially the whole way around the periphery of the recess to define a larger open mouth than the recess described with respect of the first embodiment. The side walls 121, 122 of the recess 120 are steeper than in the first embodiment. In this embodiment the length of the recess is 7.60 mm, the width 4.5 mm and the depth 1.4 mm providing a volume of 27.76 mm³.

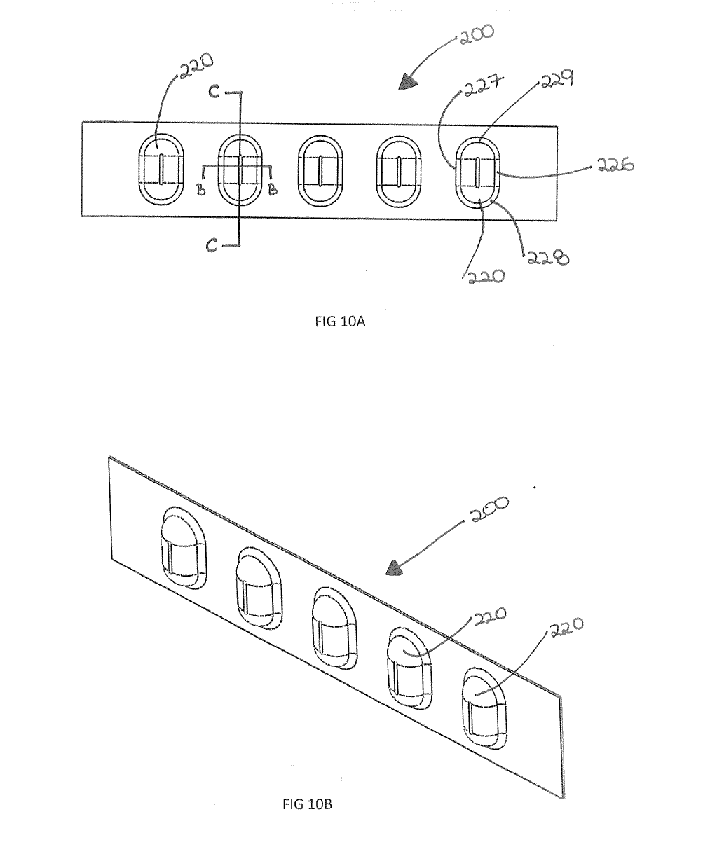

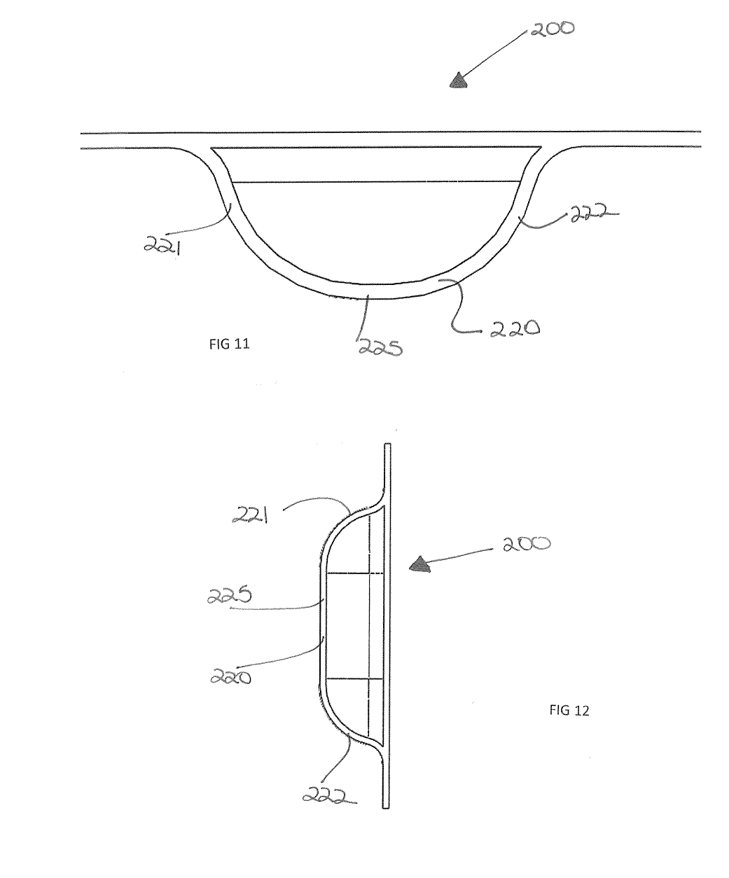

In a third embodiment illustrated in FIGS. 10 to 12, the blister strip 200 has a larger recess 220 without a shelf in the wall 221, 222 of the recess. The recess 220 has a smoothly curved wall structure 221, 222 and a flat base 225 to define a mouth with parallel straight sides 226, 227 with curved ends 228, 229 as shown in FIG. 104. In this embodiment the length of the recess is 6.8 mm, the width 4.2 mm and the depth 1.4 mm giving a volume of 24.38 mm³. Thus the volume of the three embodiments range from about 24 mm³ to 28 mm³.

The blister strip described above is specifically designed so that it can be opened by either a peeling or cutting action to allow the powder to be evacuated through use of the inhaler. Where the blister strip is designed to be opened by cutting, the order of preference for the three embodiments is the first, then the second and then the third embodiment. The first embodiment requires the least cutting force to open the recess whilst a greater cutting force is required in the second embodiment of FIGS. 5 to 9 and a still greater cutting force is required in the third embodiment of FIGS. 10 to 12. In a situation where the recess is to be opened by a peeling action then the third embodiment is viewed as the most preferable before the second embodiment with the first embodiment the least preferable. The third embodiment is the easiest to manufacture and the first embodiment is the most difficult.

The overall depth of the recess 20 is dependent on the base material used. Thus, if the base material is an aluminium laminate construction the depth is limited to a maximum of six times the overall thickness. On the other hand, if the base material is a PVC laminate construction the depth is limited to a maximum of eighteen times the overall thickness, if there is an appropriate temperature and vacuum assistance incorporated in the forming tool.

The maximum recess depth is achieved by using multiple forming operations to avoid over stretching and creating points of weakness in the base material. The minimum gap between the forming tool and its die is the thickness of the base material. For aluminium laminate constructions the maximum angle between the forming tool and die is typically 0.5 radians.

Increasing the size and shape of the cavities provides a number of advantages when the cavities are filled using a dose delivery machine that delivers an exact dose of medicament. This dose is normally between 12.5 and 14.0 mg with a volume displacement of between about 12-14 mm³ or more specifically 12.5 mm³ to 14.0 mm³ 12.5 and 14.0 mm³. The enlarged mouth of the pocket assists in ensuring that the exact dose is fed to the cavity and does not spill around the edges of the cavity. The enlarged mouth also provides a better opportunity for the air within the cavity to escape prior to the entry of the medicament powder. The fact that the powder does not totally fill the cavity is also an important feature because it includes a small amount of air that improves the aeration of the powder which in turn makes the cutting of the cavity easier and ensures a more efficient release of the powder by avoiding the powder assuming dense clumps in the base of the cavity. The use of the cavity described above in conjunction with an accurate dose delivering machine can save up to 20% in medicament. It also ensures that each cavity is much more accurately filled and provides considerably more accurate dose delivery.

The embodiments described above provide a means in which the size and shape of the cavity and spacing between cavities on the blister strip is optimized to allow for the precise use of accurate filling systems without impacting on the overall length, width and depth of the blister strip. The blister design is a simple and cost effective means by which when combined with a suitable accurate and consistent filling system, the variation in fill weight of blister strip is improved so that the goal of a reduction in dose delivery variability can be achieved.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It is to be understood that, if any prior art publication is referred to herein, such reference does not constitute an admission that the publication forms a part of the common general knowledge in the art, in Australia or any other country.

1-9. (canceled)

10. A blister pack for containing medicament in powder form for use within an inhalation device, the blister pack comprising an elongate strip of base material containing spaced recesses along the length of the strip, each recess having walls defining a cavity, and a lid hermetically sealed onto the base material to close the cavities and define pockets for medicament powder, the blister strip being adapted to have each recess filled with a metered dose of medicament powder, each recess being elongate having its longitudinal axis extending across the strip and having longer side walls joined by a base, at least one side wall including a laterally extending supplementary cavity above the base to define a mouth, whereby the volume of each recess is adapted to be greater than the volume of the metered dose and each pocket of the blister strip is adapted to be opened by either pulling back the lid or puncturing the lid or the walls of the recess.

11. The blister pack according to claim 10 wherein the mouth of each recess has a greater cross sectional area than the base.

12. The blister pack according to claim 10 wherein the supplementary cavity is provided on each side wall defining a larger mouth and a larger cavity without increasing the depth of the cavity.

13. The blister pack according to claim 10 wherein the supplementary cavity above the base extends around the periphery of the recess.

14. The blister pack according to claim 10 wherein the volume of each recess is between 1.5 and 2.5 times the volume of the metered medicament.

15. The blister pack according to claim 14 wherein the volume of each recess varies between 24 mm³ to 28 mm³.

16. The blister pack according to claim 10 wherein the pack contains between 60 and 100 sealed cavities.

17. The blister pack according to claim 10 wherein the base material is a metal foil or polymer laminate and each recess is pressed into the base material, the lid is PVC coated with adhesive to secure the lid to the base material to hermetically seal each pocket.

18. A blister pack for containing medicament in powder form for use within an inhalation device, the blister pack comprising an elongate strip of base material containing spaced recesses along the length of the strip, each recess having walls defining a cavity, and a lid hermetically sealed onto the base material to close the cavities and define pockets for medicament powder, the blister strip being adapted to have each recess filled with a metered dose of medicament powder, each recess being elongate having its longitudinal axis extending across the strip and having longer side walls joined by a base, at least one side wall including a laterally extending supplementary cavity above the base to define a mouth, whereby the volume of each recess is adapted to be greater than the volume of the metered dose and each pocket of the blister strip is adapted to be opened by either pulling back the lid or puncturing the lid or the walls of the recess and wherein the mouth of each recess has a greater cross sectional area than the base and wherein the supplementary cavity is provided on each side wall defining a larger mouth and a larger cavity without increasing the depth of the cavity.

19. The blister pack according to claim 18 wherein the supplementary cavity above the base extends around the periphery of the recess.
